(19) United States
(12) Patent Application Publication
(10) Pub. No.: US 2026/0069167 A1
(43) Pub. Date: Mar. 12, 2026

(54) Earbud Accelerometer Orientation

(71) Applicant: Samsung Electronics Co., Ltd., Suwon-si (KR)

(72) Inventors: Md Mahbubur Rahman, San Jose, CA (US); Yunzhi Li, Cupertino, CA (US); Li Zhu, Saratoga, CA (US); Mehrab Bin Morshed, Gilroy, CA (US); Jilong Kuang, San Jose, CA (US)

(21) Appl. No.: 19/321,743

(22) Filed: Sep. 8, 2025

Related U.S. Application Data
(60) Provisional application No. 63/692,635, filed on Sep. 9, 2024.

(51) Int. Cl. A61B 5/11; A61B 5/00

(52) U.S. Cl. CPC A61B 5/1102; A61B 5/1116; A61B 5/6817; A61B 5/7253; A61B 2560/0462; A61B 2562/0219; A61B 2562/0247

(57) ABSTRACT

In one embodiment, a method includes accessing an accelerometer signal from an accelerometer of an earbud worn by a wearer and determining an orientation adjustment for the accelerometer that corrects accelerometer misalignment of the earbud as worn by the wearer. The method further includes determining, based at least on the orientation adjustment for the accelerometer, an oriented accelerometer signal that represents an accelerometer signal from an optimal orientation of the earbud and determining, from the oriented accelerometer signal, a ballistocardiogram (BCG) signal of the wearer.

### PRIORITY CLAIM

This application claims the benefit under 35 U.S.C. § 119 of U.S. Provisional Patent Application No. 63/692,635 filed Sep. 9, 2024 which is incorporated by reference herein.

### TECHNICAL FIELD

This application generally relates to earbud accelerometer orientation.

### BACKGROUND

Ballistocardiogram (BCG) is a non-invasive technique that measures the mechanical recoil forces of the human body in response to cardiac blood ejection. BCG is useful for estimating important biomarkers like heart rate and heart rate variability (HRV) biomarkers, and has applications in downstream tasks such as stress monitoring. Studies also show that BCG morphological features, such as the timing and amplitude of important fiducial points of the I-J-K complex (similar to the ECG QRS complex), offer useful insights into key cardiovascular parameters, including stroke volume, cardiac output, and blood pressure.

A BCG signal can be estimated by output of an accelerometer in a wearable device, such has a pair of earbuds. However, earbuds can have varying orientation when worn, which affects the quality of biomarkers extracted from the accelerometer signal, including biomarkers for BCG signals. The alignment of the earbuds' accelerometers axes while worn has a significant effect on BCG morphology, fidelity, and variability across individuals and even across measurements, compromising the accuracy of ensuing biomarker estimates.

### BRIEF DESCRIPTION OF THE DRAWINGS

FIG. 1 illustrates an example method for correcting the orientation of an earbud worn by a wearer.

FIG. 2 illustrates an example embodiment of the method of FIG. 1 that utilizes a calibration technique.

FIG. 3 illustrates an example embodiment of the method of FIG. 1 in which an orientation adjustment is based on fusing accelerometer data from separate axes in the accelerometer's frame of reference.

FIG. 4 illustrates an example implementation of the method of FIG. 1 that fuses different types of sensor input to determine earbud orientation and placement.

FIG. 5 illustrates an example earbud having a stabilizer and several sensors.

FIG. 6 illustrates an example computing system.

### DESCRIPTION OF EXAMPLE EMBODIMENTS

Existing systems for detecting BCG signals typically obtain signals from a single accelerometer axis aligned with the head-to-foot direction, similar to traditional bed- and table-based systems. However, ear-worn devices are prone to orientation misalignment with respect to the body. Furthermore, owing to the three-dimensional nature of ballistocardiogram (BCG), the alignment of an earbuds' accelerometers axes while worn has a significant effect on BCG morphology, fidelity, and variability across individuals and even across measurements, compromising the accuracy of ensuing biomarker estimates.

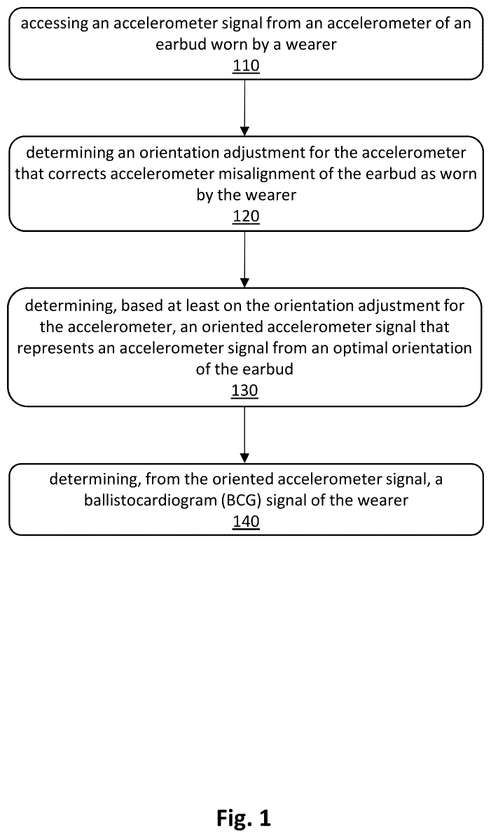

FIG. 1 illustrates an example method for correcting the orientation of an earbud worn by a wearer, thereby improving the accelerometer signals used for BCG detection and biomarker extraction. Step 110 of the example method of FIG. 1 includes accessing an accelerometer signal from an accelerometer of an earbud worn by a wearer. In particular embodiments, each of a pair of earbuds may contain an accelerometer, and the steps of example method of FIG. 1 may be performed for each earbud.

Step 120 of the example method of FIG. 1 includes determining an orientation adjustment for the accelerometer that corrects accelerometer misalignment of the earbud as worn by the wearer. Step 130 of the example method of FIG. 1 includes determining, based at least on the orientation adjustment for the accelerometer, an oriented accelerometer signal that represents an accelerometer signal from an optimal orientation of the earbud. As a result, the misalignment of the earbud in the wearer's ear is corrected, and therefore the orientation of the accelerometer and its output signals is optimized for determining the wearer's BCG signals from the accelerometer signals. In particular embodiments, step 120 may precede step 110, and vice versa.

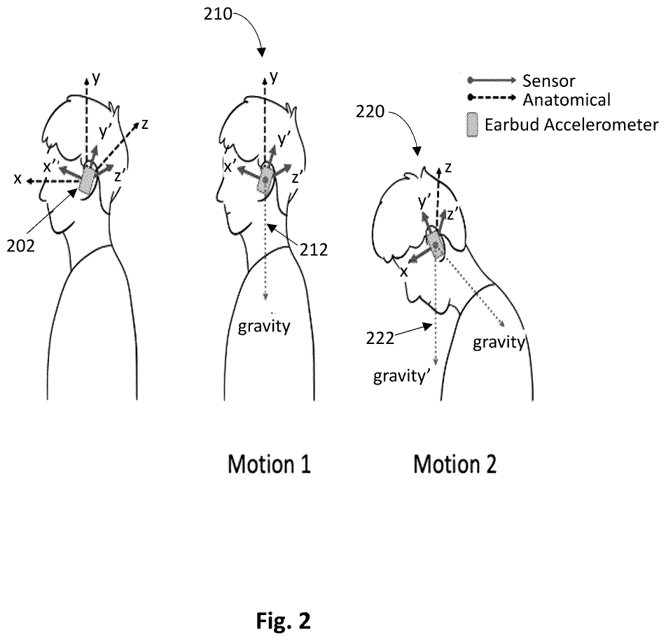

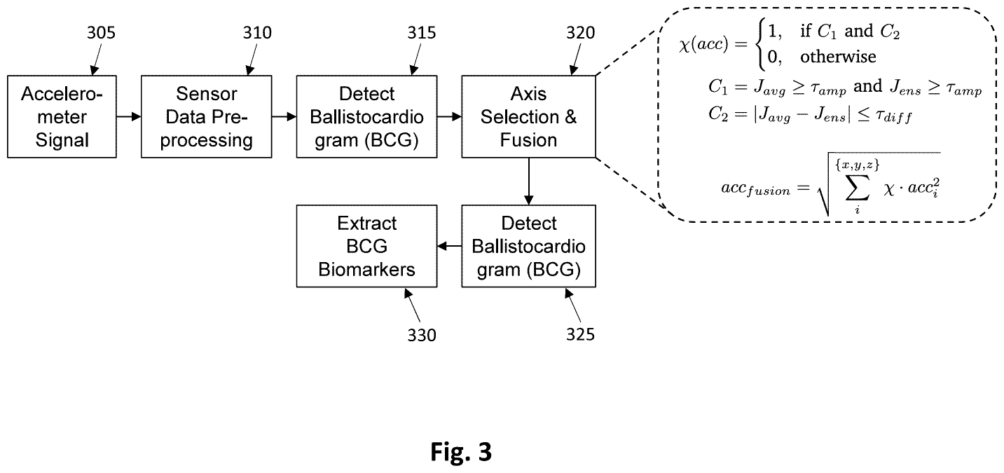

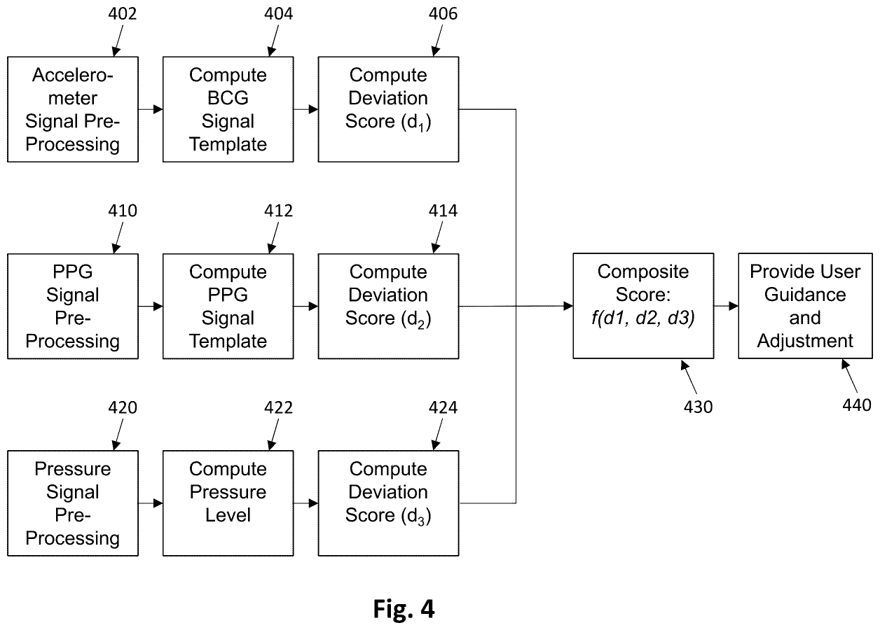

FIGS. 2-4 provide example, non-exhaustive implementations of the example of FIG. 1. For instance, FIG. 1 may be implemented using a calibration technique, such as illustrated in FIG. 2, in which the calibration determines the orientation adjustment (i.e., the orientation adjustment correcting for misalignment of the earbud) and then outputs a corrected (oriented) accelerometer signal, which approximates the accelerometer signal that would occur if the earbud was properly oriented. As another example, FIG. 3 illustrates an embodiment in which the orientation adjustment is determined based on BCG signal quality from signals about individual accelerometer axes, and the oriented accelerometer signal is a fusion of the individual signals (or the single best signal if fusion does not occur). Finally, FIG. 4 illustrates an embodiment in which the orientation adjustment is based on a signal deviation for several sensor signals, including an accelerometer, and the oriented accelerometer signal is the signal obtained after prompted or guided earbud adjustments made by the wearer.

FIG. 2 illustrates an example technique implementing steps 130 and 140 of the example of FIG. 1. The implementation of FIG. 2 illustrates an example of a calibration procedure involving user head poses or motions to determine the orientation adjustment for an earbud accelerometer. For instance, the example of FIG. 2 involves two static calibration poses to determine a rotation matrix between the accelerometer 202's local coordinate frame and the subject's anatomical reference frame, resulting in identification of the acceleration along the wearer's head-to-foot axis even when sensor misalignment occurs due to how the earbuds are worn (i.e., even when no accelerometer axis aligns with the head-to-foot axis as worn by the wearer).

In the first pose 210, the user stands or sits upright for a few seconds. The gravity vector vy and the head-to-foot axis is then determined by:

$${v}_{y} = - {v}_{gravity} = {[ \frac{- {acc}_{x}}{{acc}_{mag}} , \frac{- {acc}_{y}}{{acc}_{mag}} , \frac{- {acc}_{z}}{{acc}_{mag}} ]}^{T} \qquad ( 1 )$$

where accₓ, acc_{y}, acc_{z} are the current 3-axis accelerometer value upright position, acc_{mag} is the L2-norm of the 3-axis, and v_{gravity} is the normalized vector representing the gravity vector while the user is in pose 210. In the second pose 220, the wearer tilts their head forward about the transverse axis (z-axis), and the new gravity vector v_{gravity′} is then determined using equation (1) in the new pose. Then, the transverse axis is the cross product of the two gravity vectors:

$${v}_{z} = {v}_{gravity} \times {v}_{{gravity}^{′}} ; {v}_{x} = {v}_{y} \times {v}_{z} \qquad ( 2 )$$

and the frontal axis (x-axis) is determined by the cross product of the head-to-foot and transverse axes, as shown in Eqn. 2. These vectors form the rotation matrix that aligns the accelerometer with the subject's anatomical frame. Using this matrix, the implementation of FIG. 2 transforms and isolates the calibrated acceleration, i.e., the acceleration along the wearer's head-to-foot axis, as:

$${acc}_{calibrated} = {[ {v}_{x} , {v}_{y} , {v}_{z} ]}^{T} \cdot {acc}_{earbud} \qquad ( 3 )$$

where acc_{earbud} is the current accelerometer value in any head orientation after calibration and acc_{calibrated} is the orientation aligned accelerometer data. This calibrated accelerometer data can then be used to, for example, determine a BCG signal and extract related biomarkers.

The implementation of FIG. 2 may be repeated periodically. For example, each time a person inserts or adjusts the earbuds (as detected by one or more sensors on the earbuds, e.g., an IMU, a light sensor, a pressure sensor, a capacitive sensor, etc.), then the calibration procedure may be repeated in order to calibrate the earbuds. In particular embodiments, the calibration procedure may be performed before acquiring accelerometer data that is used to estimate the wearer's BCG signals.

While the example of FIG. 2 illustrates two particular poses to use to calibrate the earbud accelerometer, this disclosure contemplates that more or fewer poses or motions may be used, and other poses may likewise be used to perform a calibration procedure to determine the orientation adjustment for the earbud accelerometer. In particular embodiments, a device (e.g., the earbuds or a connected device such as a smartphone or a TV, etc.) may instruct the user during the calibration procedure, e.g., by showing on a display of a smartphone the poses for the user to take, or by verbally instruction the user via the headphone speakers, etc.

FIG. 3 illustrates an example embodiment in which an orientation adjustment is based on fusing (or selecting) accelerometer data from separate axes in the accelerometer's frame of reference. Unlike the approach of FIG. 2, the example approach of FIG. 3 does not require active participation from the wearer (e.g., during a calibration phase), and therefore this technique can be less intrusive for the wearer. In addition, regardless of the orientation of the earbud inside the wearer's ear, this technique can assess the strength of the detected BCG signal individually from the 3 accelerometer axes and then determine whether one or more of those signals are of sufficient quality to use for subsequent BCG detection and biomarker extraction.

The example technique of FIG. 3 includes accessing an accelerometer signal 305, performing pre-processing 310, and then detecting a BCG signal 315 from the signal associated with each accelerometer axis. Axis selection and fusion step 320 determines any adjustments to the accelerometer signal on a per axis basis. First, step 320 includes determining two metrics from the detected BCG data from each axis (which may be, e.g., a 30-second window of data): (1) the average J-peak amplitude within the window of data, denoted as J_{avg}, and (2) the J-peak amplitude of the ensemble-averaged BCG waveform (the average of all the BCG waveforms overlayed centered at J-peaks) within the same window, denoted as Jₑₙₛ.

If the quality of the BCG signal is of good quality over the window, the J-peak amplitude will be significantly higher compared to surrounding peaks and the difference between J_{avg} and Jₑₙₛ will be very low. Therefore, step 320 includes assessing BCG signal quality for each axis using two thresholds, τₐₘₚ and τ_{diff}, which balance data yield and signal quality. Sensitivity analysis of the amplitude threshold, τₐₘₚ, shows that lowering τₐₘₚ increases data yield by admitting low-quality signals, while raising it above 6 mG significantly reduces yield where G is the Earth's gravitational acceleration. Similarly, lowering τ_{diff} improves signal quality but decreases yield, while increasing it beyond 2 mG introduces noise into the signal. Therefore, in particular embodiments τₐₘₚ may have a value of 4 mG to ensure sufficient signal strength, while incorporating considerations based on typical BCG amplitudes (10 mG) in the head. The difference threshold, τ_{diff}, may have a value of 2 mG to exclude axes with excessive discrepancies between Jₑₙₛ and Jav_{g}. These thresholds are based on the accelerometer having a +/−2G sensitivity, and other thresholds may be used in other embodiments. The oriented accelerometer signal acc_{fusion} may then be determined as:

$$x ( acc ) = \{ \begin{matrix}1 , & if {C}_{1} and {C}_{2} \\ 0 , & otherwise\end{matrix}$$

$${C}_{1} = {J}_{avg} \geq {τ}_{amp} and {J}_{ens} \geq {τ}_{amp}$$

$${C}_{2} = \vert {J}_{avg} - {J}_{ens} \vert \leq {τ}_{diff}$$

$${acc}_{fusion} = \sqrt{{\sum }_{i}^{\{ x , y , z \}} x \cdot {acc}_{i}^{2}}$$

where X is the indicator function, acci is the accelerometer value and accfusioₙ is the orientation-adjusted accelerometer data for accurate BCG biomarker extraction. While the example of FIG. 3 uses two particular BCG signal attributes (average J-peak amplitude and the J-peak amplitude of the ensemble-averaged BCG waveform) this disclosure contemplates that other attributes and/or more or fewer attributes along with corresponding quality thresholds may be used in a fusion procedure.

As explained above, in particular embodiments the technique of FIG. 3 may fuse accelerometer signals from distinct axes, provided those signals meet the quality threshold for resulting BCG signals. However, if only one signal meets the quality threshold, then the fusion technique of FIG. 3 performs selection by choosing that particular signal to use to subsequently determine BCG signals (i.e., the selected signal is a good approximation of a signal aligned with the head-to-foot orientation).

Particular embodiments may use both a calibration technique (e.g., as in the example of FIG. 2) and a calibration-free technique (e.g., as in the example of FIG. 3) to generate more accurate biomarkers from an earbud accelerometer BCG signal. For example, accelerometer data may be obtained and then processed (e.g., by filtering (e.g., a band-pass filter) to remove noise from the data but preserve the IJK complex-like morphology in the signal). The quality of the BCG signal determined from the accelerometer data may optionally be determined by applying further signal processing or machine learning algorithms on extracted time and frequency domain features on a specific time window (e.g., 30-seconds) of BCG signal. Then, a calibration procedure such as in FIG. 2 may be used to obtain an oriented accelerometer signal. Even then, due to slight variations of the head or body posture, axis fusion may still produce better biomarkers compared to a single axis. As a result, a BCG signal may be determined about each accelerometer axis, as in the example of FIG. 3, and then axis fusion and selection may be performed to arrive a fully calibrated accelerometer data for BCG signal detection and biomarker extraction.

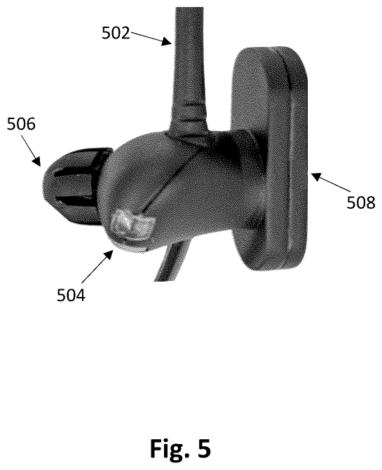

Particular embodiments may capture sensor signals from different anatomical sites inside the ear and fuse these signals together to determine earbud orientation, as well as earbud fit in particular embodiments. FIG. 4 illustrates an example implementation that fuses different types of sensor input to determine earbud orientation and placement. In particular embodiments, the implementation of FIG. 4 may use an earbud such as illustrated in FIG. 5, which illustrates an earbud having a stabilizer 502 and several sensors, including a temperature sensitive speaker 506, an accelerometer and/or gyroscope 508 (which is oriented along the wearer's head-to-foot axis), a microphone, and a PPG sensor and LED 504. The expected orientation of the earbud is that the Y-axis of the accelerometer/gyroscope will be aligned with the head-to-foot body axis of the wearer. With this expected orientation, the stabilizer's top touches the concha area or the stabilizer-like extended part of the earbud can be touching another part of the ear including the back of the ear, and PPG sensor senses the blood flow around the canal area. In an alternative embodiment, the PPG sensor can be part of the stabilizerlike extended part of the earbud touching the front or back of the ear capturing the blood flow around the ear.

The example of FIG. 4 uses accelerometer, photoplethysmography (PPG), and pressure sensors to align the earbud inside the ear to get more accurate biomarkers. Step 402 takes the accelerometer signal and filters out the noise using, e.g., a bandpass filter from the Y-axis accelerometer signal to remove high frequency noise and baseline wander. Step 404 detects each heart beat in the signal by calculating the probability of all potential inter-beat-intervals (IBI) in a specific window (e.g., 30 seconds) and selecting the highest probability. It then detects each BCG beat based on the estimated IBI and amplitude of the J-peak inside each BCG beat. This embodiment utilizes one or more supervised best orientation and snug-fit accelerometer (BCG) signal templates to compute the deviation of the current orientation. The expected signal template can be trained on the data collected from a group of subjects with the best possible head-to-foot orientation of the device using the following formula:

$$T ( i ) = \frac{1}{K} {\sum }_{k = 1}^{K} {S}_{k} ( i ) \qquad ( 4 )$$

where T(i) is the i^{h} sample of the template T, Sₖ(i) is the i^{th} sample of the kl sensor segment (e.g., one BCG segment related to one heartbeat), K is the total number of sensor segments. Step 406 then determines the deviation d₁ due to the accelerometer signal as

$$d 1 = \frac{1}{M} {\sum }_{i = 1}^{M} ‖ {T}_{c} ( i ) - T ( i ) ‖ \qquad ( 5 )$$

where T_{c}(i) is the i^{h} sample of the current template T_{c} and M is the total number of samples.

Likewise, step 410 takes the PPG signal and filters out noise from the PPG signal. This embodiment detects PPG data segment associated with each heartbeat by detecting peaks and troughs in the PPG signal. If the earbud is in the correct orientation, then given the location of the PPG sensor on the correctly oriented earbud, the PPG sensor will capture reflected light from the ear canal, and step 412 then determines a current template from the PPG data by using Equation 4 or the like. Step 414 then determines the deviation d₂ using equation 5 above for the PPG signal morphology, where the original PPG template is determined using equation 4 from ground-truth PPG data.

The example of FIG. 4 utilizes a pressure sensor on top of the earbud stabilizer to assess the level of pressure with the expect earbud fit and orientation. Step 420 includes filtering out the noise by using median filter. Step 422 determines the current pressure level, and step 424 then computes the deviation (d₃) of the pressure based on the difference in the current pressure and the expected pressure level, where the expected pressure is trained from a set of subjects' data including variations of ear size, shape, skin color, age, gender, and other demographic variables.

After each deviation is determined, then the deviations (d₁, d₂, d₃) are used in step 430 to compute a composite deviation score as a function of these three deviations, e.g., as a weighted sum of those deviations, for example. Particular embodiments can also determine weights for each deviation based on their importance in terms of capturing more accurate biomarkers. For example, for pulse transit time (PTT) estimation, this embodiment can put the highest weight on d₁ followed by d₂ and d₃. Another embodiment can ignore d₃ by putting its weight=0 due to potential discomfort of the user. Another embodiment may consider more sensor modalities and can calculate the deviation of the earbud orientation from the expected orientation by different combinations of d₁, d₂, d₃, . . . , dₙ.

Then, step 440 includes guiding the wearer in real-time to adjust the earbud orientation for more accurate biomarker measurements. For example, a connected smartphone display can show a quantification of how close the three or more signal metrics are when the wearer adjusts their earbud inside the ear. Other embodiments may provide guidance via the earbud. Particular embodiments may instruct the user regarding a particular earbud position to use or about particular steps to put the earbud into a particular position that is closer to the earbud's optimal orientation.

Step 140 of the example method of FIG. 1 includes determining, from the oriented accelerometer signal, a BCG signal of the wearer. Biomarkers may then be extracted from the BCG signal, such as for example stroke volume, cardiac output, total peripheral resistance, and blood pressure, to name a few. The techniques of this disclosure improve the quality of the resulting BCG signal and extracted biomarkers based on earbud accelerometer data.

In particular embodiments, the example method of FIG. 1 may be performed only when the earbud is detected to be worn by a wearer, e.g., based on a sensor signal (e.g., pressure, light temperature, etc.) or based on other indications of the earbud's location (e.g., whether it is in a charging case).

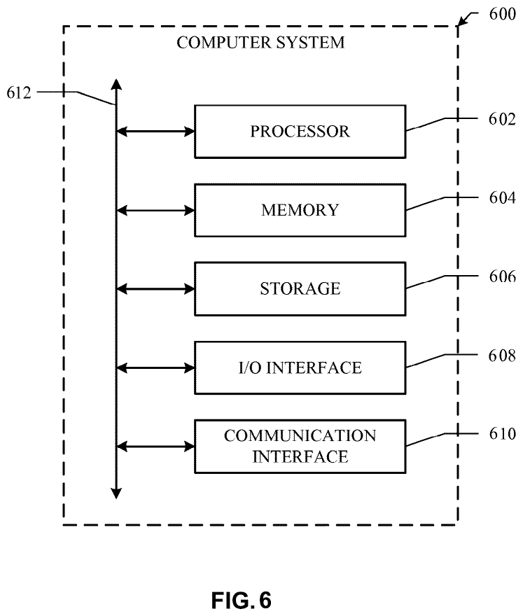

FIG. 6 illustrates an example computer system 600. In particular embodiments, one or more computer systems 600 perform one or more steps of one or more methods described or illustrated herein. In particular embodiments, one or more computer systems 600 provide functionality described or illustrated herein. In particular embodiments, software running on one or more computer systems 600 performs one or more steps of one or more methods described or illustrated herein or provides functionality described or illustrated herein. Particular embodiments include one or more portions of one or more computer systems 600. Herein, reference to a computer system may encompass a computing device, and vice versa, where appropriate. Moreover, reference to a computer system may encompass one or more computer systems, where appropriate.

This disclosure contemplates any suitable number of computer systems 600. This disclosure contemplates computer system 600 taking any suitable physical form. As example and not by way of limitation, computer system 600 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 600 may include one or more computer systems 600; be unitary or distributed; span multiple locations; span multiple machines; span multiple data centers; or reside in a cloud, which may include one or more cloud components in one or more networks. Where appropriate, one or more computer systems 600 may perform without substantial spatial or temporal limitation one or more steps of one or more methods described or illustrated herein. As an example and not by way of limitation, one or more computer systems 600 may perform in real time or in batch mode one or more steps of one or more methods described or illustrated herein. One or more computer systems 600 may perform at different times or at different locations one or more steps of one or more methods described or illustrated herein, where appropriate.

In particular embodiments, computer system 600 includes a processor 602, memory 604, storage 606, an input/output (I/O) interface 608, a communication interface 610, and a bus 612. Although this disclosure describes and illustrates a particular computer system having a particular number of particular components in a particular arrangement, this disclosure contemplates any suitable computer system having any suitable number of any suitable components in any suitable arrangement.

In particular embodiments, processor 602 includes hardware for executing instructions, such as those making up a computer program. As an example and not by way of limitation, to execute instructions, processor 602 may retrieve (or fetch) the instructions from an internal register, an internal cache, memory 604, or storage 606; decode and execute them; and then write one or more results to an internal register, an internal cache, memory 604, or storage 606. In particular embodiments, processor 602 may include one or more internal caches for data, instructions, or addresses. This disclosure contemplates processor 602 including any suitable number of any suitable internal caches, where appropriate. As an example and not by way of limitation, processor 602 may include one or more instruction caches, one or more data caches, and one or more translation lookaside buffers (TLBs). Instructions in the instruction caches may be copies of instructions in memory 604 or storage 606, and the instruction caches may speed up retrieval of those instructions by processor 602. Data in the data caches may be copies of data in memory 604 or storage 606 for instructions executing at processor 602 to operate on; the results of previous instructions executed at processor 602 for access by subsequent instructions executing at processor 602 or for writing to memory 604 or storage 606; or other suitable data. The data caches may speed up read or write operations by processor 602. The TLBs may speed up virtual-address translation for processor 602. In particular embodiments, processor 602 may include one or more internal registers for data, instructions, or addresses. This disclosure contemplates processor 602 including any suitable number of any suitable internal registers, where appropriate. Where appropriate, processor 602 may include one or more arithmetic logic units (ALUs); be a multi-core processor; or include one or more processors 602. Although this disclosure describes and illustrates a particular processor, this disclosure contemplates any suitable processor.

In particular embodiments, memory 604 includes main memory for storing instructions for processor 602 to execute or data for processor 602 to operate on. As an example and not by way of limitation, computer system 600 may load instructions from storage 606 or another source (such as, for example, another computer system 600) to memory 604. Processor 602 may then load the instructions from memory 604 to an internal register or internal cache. To execute the instructions, processor 602 may retrieve the instructions from the internal register or internal cache and decode them. During or after execution of the instructions, processor 602 may write one or more results (which may be intermediate or final results) to the internal register or internal cache. Processor 602 may then write one or more of those results to memory 604. In particular embodiments, processor 602 executes only instructions in one or more internal registers or internal caches or in memory 604 (as opposed to storage 606 or elsewhere) and operates only on data in one or more internal registers or internal caches or in memory 604 (as opposed to storage 606 or elsewhere). One or more memory buses (which may each include an address bus and a data bus) may couple processor 602 to memory 604. Bus 612 may include one or more memory buses, as described below. In particular embodiments, one or more memory management units (MMUs) reside between processor 602 and memory 604 and facilitate accesses to memory 604 requested by processor 602. In particular embodiments, memory 604 includes random access memory (RAM). This RAM may be volatile memory, where appropriate Where appropriate, this RAM may be dynamic RAM (DRAM) or static RAM (SRAM). Moreover, where appropriate, this RAM may be single-ported or multi-ported RAM. This disclosure contemplates any suitable RAM. Memory 604 may include one or more memories 604, where appropriate. Although this disclosure describes and illustrates particular memory, this disclosure contemplates any suitable memory.

In particular embodiments, storage 606 includes mass storage for data or instructions. As an example and not by way of limitation, storage 606 may include a hard disk drive (HDD), a floppy disk drive, flash memory, an optical disc, a magneto-optical disc, magnetic tape, or a Universal Serial Bus (USB) drive or a combination of two or more of these. Storage 606 may include removable or non-removable (or fixed) media, where appropriate. Storage 606 may be internal or external to computer system 600, where appropriate. In particular embodiments, storage 606 is non-volatile, solid-state memory. In particular embodiments, storage 606 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 606 taking any suitable physical form. Storage 606 may include one or more storage control units facilitating communication between processor 602 and storage 606, where appropriate. Where appropriate, storage 606 may include one or more storages 606. Although this disclosure describes and illustrates particular storage, this disclosure contemplates any suitable storage.

In particular embodiments, I/O interface 608 includes hardware, software, or both, providing one or more interfaces for communication between computer system 600 and one or more I/O devices. Computer system 600 may include one or more of these I/O devices, where appropriate. One or more of these I/O devices may enable communication between a person and computer system 600. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 608 for them. Where appropriate, I/O interface 608 may include one or more device or software drivers enabling processor 602 to drive one or more of these I/O devices. I/O interface 608 may include one or more I/O interfaces 608, where appropriate. Although this disclosure describes and illustrates a particular I/O interface, this disclosure contemplates any suitable I/O interface.

In particular embodiments, communication interface 610 includes hardware, software, or both providing one or more interfaces for communication (such as, for example, packet-based communication) between computer system 600 and one or more other computer systems 600 or one or more networks. As an example and not by way of limitation, communication interface 610 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 610 for it. As an example and not by way of limitation, computer system 600 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 600 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 600 may include any suitable communication interface 610 for any of these networks, where appropriate. Communication interface 610 may include one or more communication interfaces 610, where appropriate. Although this disclosure describes and illustrates a particular communication interface, this disclosure contemplates any suitable communication interface.

In particular embodiments, bus 612 includes hardware, software, or both coupling components of computer system 600 to each other. As an example and not by way of limitation, bus 612 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 612 may include one or more buses 612, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

This disclosure contemplates a system that includes one or more non-transitory computer readable storage media storing instructions; and one or more processors coupled to the one or more non-transitory computer readable storage media and operable to execute the instructions to perform certain functions includes embodiments in which those functions are performed by a single processor, embodiments in which those functions are performed by multiple processors that each perform all the functions, and embodiments in which those functions are performed by multiple processors (e.g., in separate computing devices) where each processor performs at least one function but less than all recited functions.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend.

What is claimed is:

1. A method comprising:
accessing an accelerometer signal from an accelerometer of an earbud worn by a wearer;
determining an orientation adjustment for the accelerometer that corrects accelerometer misalignment of the earbud as worn by the wearer;
determining, based at least on the orientation adjustment for the accelerometer, an oriented accelerometer signal that represents an accelerometer signal from an optimal orientation of the earbud; and
determining, from the oriented accelerometer signal, a ballistocardiogram (BCG) signal of the wearer.

2. The method of claim 1, wherein:
determining the orientation adjustment for the accelerometer comprises determining, from a calibration procedure comprising a plurality of poses of the wearer, a rotation matrix that aligns the accelerometer with the wearer's anatomical frame; and
determining the oriented accelerometer signal comprises transforming the accessed accelerometer signal using the rotation matrix that aligns the accelerometer with the wearer's anatomical frame.

3. The method of claim 2, wherein the calibration procedure comprises:
a first pose in which the wearer is upright; and
a second pose in which the wearer tilts the wearer's head, from the first pose, about a transverse axis.

4. The method of claim 1, wherein:
determining the orientation adjustment for the accelerometer comprises:
determining, from the accessed accelerometer signal, a BCG signal about each accelerometer axis;
determining, for each BCG signal and from one or more attributes of each BCG signal, whether a BCG signal quality exceeds a quality threshold; and
determining the oriented accelerometer signal comprises (1) for each accelerometer axis, selecting that accelerometer axis when the corresponding BCG signal exceeds the quality threshold and (2) fusing signals from each selected accelerometer axes to create the oriented accelerometer signal.

5. The method of claim 4, wherein:
the one or more attributes comprise (1) an average J-peak amplitude within the BCG signal, and (2) a J-peak amplitude of an ensemble-averaged BCG waveform; and
the quality threshold comprises each attribute having a value that meets or exceeds a corresponding threshold value.

6. The method of claim 5, wherein the quality threshold comprises a first threshold τₐₘₚ having a value of about 4 mG and a second threshold τ_{diff} having a value of about 2 mG.

7. The method of claim 1, wherein determining an orientation adjustment for the accelerometer that corrects accelerometer misalignment of the earbud as worn by the wearer comprises:
comparing the accessed accelerometer signal to a template accelerometer signal corresponding to the optimal orientation;
determining, based on the comparison, an accelerometer deviation score for the accessed accelerometer signal; and
determining an orientation of the earbud based on the accelerometer deviation score.

8. The method of claim 7, further comprising:
accessing a PPG signal from a PPG sensor of the earbud;
accessing a pressure level from a pressure sensor of the earbud;
comparing (1) the accessed PPG signal to a template PPG signal corresponding to the optimal orientation and (2) the accessed pressure level to an expected pressure level corresponding to the optimal orientation;
determining a PPG deviation score and a pressure-level deviation score based on the comparisons; and
determining the orientation of the earbud based on a combination of the accelerometer deviation score, the PPG deviation score, and the pressure-level deviation score.

9. The method of claim 8, further comprising providing feedback to the wearer regarding the determined earbud orientation.

10. The method of claim 1, wherein determining an orientation adjustment for the accelerometer that corrects accelerometer misalignment of the earbud as worn by the wearer comprises:
comparing an accessed PPG signal from a PPG sensor of the earbud to a template PPG signal corresponding to the optimal orientation;
determining, based on the comparison, a PPG deviation score for the accessed PPG signal; and
determining an orientation of the earbud based on the PPG deviation score.

11. The method of claim 1, wherein determining an orientation adjustment for the accelerometer that corrects accelerometer misalignment of the earbud as worn by the wearer comprises:
comparing an accessed pressure data from a pressure sensor of the earbud to an expected pressure data corresponding to the optimal orientation;
determining, based on the comparison, a pressure deviation score for the accessed pressure data; and
determining an orientation of the earbud based on the PPG deviation score.

12. A system comprising one or more non-transitory computer readable storage media storing instructions; and one or more processors coupled to the one or more non-transitory computer readable storage media and operable to execute the instructions to:
access an accelerometer signal from an accelerometer of an earbud worn by a wearer;
determine an orientation adjustment for the accelerometer that corrects accelerometer misalignment of the earbud as worn by the wearer;
determine, based at least on the orientation adjustment for the accelerometer, an oriented accelerometer signal that represents an accelerometer signal from an optimal orientation of the earbud; and
determine, from the oriented accelerometer signal, a ballistocardiogram (BCG) signal of the wearer.

13. The system of claim 11, wherein:
determining the orientation adjustment for the accelerometer comprises determining, from a calibration procedure comprising a plurality of poses of the wearer, a rotation matrix that aligns the accelerometer with the wearer's anatomical frame; and
determining the oriented accelerometer signal comprises transforming the accessed accelerometer signal using the rotation matrix that aligns the accelerometer with the wearer's anatomical frame.

14. The system of claim 12, wherein the calibration procedure comprises:
a first pose in which the wearer is upright; and
a second pose in which the wearer tilts the wearer's head, from the first pose, about a transverse axis.

15. The system of claim 11, wherein:
determining the orientation adjustment for the accelerometer comprises:
determining, from the accessed accelerometer signal, a BCG signal about each accelerometer axis;
determining, for each BCG signal and from one or more attributes of each BCG signal, whether a BCG signal quality exceeds a quality threshold; and
determining the oriented accelerometer signal comprises (1) for each accelerometer axis, selecting that accelerometer axis when the corresponding BCG signal exceeds the quality threshold and (2) fusing signals from each selected accelerometer axes to create the oriented accelerometer signal.

16. The system of claim 14, wherein:
the one or more attributes comprise (1) an average J-peak amplitude within the BCG signal, and (2) a J-peak amplitude of an ensemble-averaged BCG waveform; and
the quality threshold comprises each attribute having a value that meets or exceeds a corresponding threshold value.

17. The system of claim 15, wherein the quality threshold comprises a first threshold τₐₘₚ having a value of about 4 mG and a second threshold τ_{diff} having a value of about 2 mG.

18. The system of claim 11, wherein determining an orientation adjustment for the accelerometer that corrects accelerometer misalignment of the earbud as worn by the wearer comprises:
comparing the accessed accelerometer signal to a template accelerometer signal corresponding to the optimal orientation;
determining, based on the comparison, an accelerometer deviation score for the accessed accelerometer signal; and
determining an orientation of the earbud based on the accelerometer deviation score.

19. The system of claim 17, further comprising one or more processors that are operable to execute the instructions to:
access a PPG signal from a PPG sensor of the earbud;
access a pressure level from a pressure sensor of the earbud;
compare (1) the accessed PPG signal to a template PPG signal corresponding to the optimal orientation and (2) the accessed pressure level to an expected pressure level corresponding to the optimal orientation;
determine a PPG deviation score and a pressure-level deviation score based on the comparisons; and
determine the orientation of the earbud based on a combination of the accelerometer deviation score, the PPG deviation score, and the pressure-level deviation score.

20. One or more non-transitory computer readable storage media storing instructions that are operable when executed by one or more processors to:
access an accelerometer signal from an accelerometer of an earbud worn by a wearer;
determine an orientation adjustment for the accelerometer that corrects accelerometer misalignment of the earbud as worn by the wearer;
determine, based at least on the orientation adjustment for the accelerometer, an oriented accelerometer signal that represents an accelerometer signal from an optimal orientation of the earbud; and
determine, from the oriented accelerometer signal, a ballistocardiogram (BCG) signal of the wearer.
